(19) United States
(12) Patent Application Publication
(10) Pub. No.: US 2012/0071693 A1
(43) Pub. Date: Mar. 22, 2012

(54) PROCESS FOR THE PRODUCTION OF ETHYLENE GLYCOL AND RELATED COMPOUNDS

(76) Inventors: Eric Johannes Maria De Boer, Amsterdam (NL); Harry Van Der Heijden, Amsterdam (NL); Harry Van Der Heijden, Amsterdam (NL); Meurs Jan Hermen Hendrik, Amsterdam (NL); Sanne Wijnans, Amsterdam (NL)

(21) Appl. No.: 13/319,853

(22) PCT Filed: May 10, 2010
(86) PCT No.: PCT/EP2010/056387

§ 371 Date: Nov. 29, 2011

(30) Foreign Application Priority Data
May 12, 2009 (EP) 09159962.1

(51) Int. Cl. C07C 29/149; C07C 209/50

(52) U.S. Cl. 564/489; 568/861; 568/864

(57) ABSTRACT

The present invention provide a process for the production of compounds of general formula (I), Y—CH₂CH₂—Z (I) wherein Y and Z are functional groups independently selected from the group consisting of a hydroxyl group and R¹R²N and wherein R¹ and R² may be the same or different and are functional groups selected from the group consisting of hydrogen and substituted or non-substituited alkyl groups comprising 1 to 8 carbon atoms, or R¹R²N is a cyclic compound selected from the group of aromatic and non-aromatic cyclic compounds optionally comprising one or more heteroatoms in addition to the nitrogen atom, said process comprising the steps of: (i) reacting carbon monoxide and an amine in the presence of oxygen to provide a compound of general formula (II) wherein R¹ and R² or R¹R²N are as defined above and X is selected from the group consisting of R¹R²N and R³O, wherein R³ is selected from alkyl groups comprising 1 to 8 carbon atoms; and (ii) converting the compound of general formula (II) into a compound of general formula (I) by a process that comprises a hydrogenation reaction.

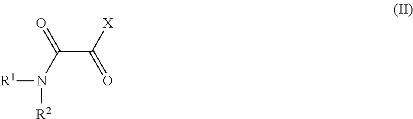

### FIELD OF THE INVENTION

This invention relates to a process for the production of ethylene glycol. It also relates to processes for the production of ethyldiamines and ethanolamines.

### BACKGROUND OF THE INVENTION

Ethylene glycol, also known as mono-ethylene glycol (MEG) is widely used as an antifreeze, e.g. in the automotive industry, and as a raw material in the manufacture of polyethylene terephthalate (PET) resin and fibres.

Ethylene glycol is generally produced from ethylene oxide (EO), which is itself produced by direct oxidation of ethylene in the presence of a silver catalyst. Conversion of EO to MEG can be carried out via hydrolysis with water under pressure or catalytic conditions. In recent years the selective synthesis of ethylene glycol via the intermediate ethylene carbonate has been described in U.S. Pat. No. 6,080,897 and U.S. Pat. No. 6,187,972. Ethylene carbonate can be obtained by reaction of ethylene oxide with carbon dioxide and can be selectively hydrolysed to form MEG in high yield.

Long-term shortage and high crude oil prices have led to intensive research into methods for the production of chemical intermediates such as MEG from C₁ units, such as synthesis gas and carbon monoxide. These C₁ materials may be obtained, for example, by the gasification of coal or biomass.

At high pressure carbon monoxide and hydrogen react directly to produce ethylene glycol, but such a process is slow, non-selective and expensive in catalyst. Other methods that have been researched involve the formation of methanol or formaldehyde and the subsequent catalytic conversion of these materials in to ethylene glycol.

The production of dimethyl oxalate via the reaction of carbon monoxide and methanol in the presence of oxygen has been described in U.S. Pat. No. 4,874,888. The resultant dimethyl oxalate can be hydrogenated to form MEG in a selective manner H. T. Teunissen and C. J. Elsevier J. Chem. Soc., Chem. Commun., 1997, 667-668. This process is complicated by the sensitivity of the oxalate intermediate to water.

Related compounds such as ethanolamines and ethyldiamines are also important industrially as chemical intermediates and chelating agents. Ethanolamine, for example, can be used as a scrubbing agent to remove carbon dioxide and hydrogen sulfide from gas streams. These compounds can be made by reaction of EO, MEG or chlorinated ethylene species with ammonia. A process for making these compounds, which process avoided the use of intermediates derived from ethylene (and crude oil) would be advantageous.

The manufacture of oxamides via the oxidative reaction of carbon monoxide with amines has been described in I. Pri-Bar and H. Alper, Can. J. Chem, 1990, 68, 1544-1547. Oxamides are much less sensitive to aqueous environments than oxalates.

### SUMMARY OF THE INVENTION

The present invention provides a process for the production of compounds of general formula (I),

Y—CH₂CH₂—Z (I)

wherein Y and Z are functional groups independently selected from the group consisting of a hydroxyl group and R¹R²N and wherein R¹ and R² may be the same or different and are functional groups selected from the group consisting of hydrogen and substituted or non-substituted alkyl groups comprising 1 to 8 carbon atoms, or R¹R²N is a cyclic compound selected from the group of aromatic and non-aromatic cyclic compounds optionally comprising one or more heteroatoms in addition to the nitrogen atom, said process comprising the steps of:
(i) reacting carbon monoxide and an amine in the presence of oxygen to provide a compound of general formula II:

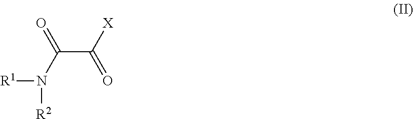

wherein R¹ and R² or R¹R²N are as defined above and X is selected from the group consisting of R¹R²N and R³⁰, wherein R³ is selected from alkyl groups comprising 1 to 8 carbon atoms; and
(ii) converting the compound of general formula (II) into a compound of general formula (I) by a process that comprises a hydrogenation reaction.

The present invention also provides a process for the preparation of ethylene glycol by the hydrogenation of an oxamide.

### DETAILED DESCRIPTION OF THE INVENTION

The present invention relates to a method suitable for the production of ethylene compounds containing a single substituent on each carbon atom. The substituents are independently selected from hydroxyl and amine groups. That is, the product ethylene compounds are selected from the group consisting of ethylene glycol, ethanolamines and ethyldiamines. The present invention advantageously provides a method for producing these compounds avoiding the use of intermediates derived from ethylene.

The process of the present invention involves a first step comprising the reaction of carbon monoxide and an amine in the presence of oxygen in order to provide a compound of general formula (II). That is an oxamide (X═R¹R²N) or an oxamate (X═OR³).

The preparation of an oxamide using this method may be carried out under any suitable conditions, such as those indicated in I. Pri-Bar and H. Alper, Can. J. Chem., 1990, 68, 1544-1547; T. Saegusa et al., Tetrahedron Lett., 1968, 13, 1641-1644; or K. Hiwatara et al., Bull. Chem. Soc. Jpn., 2004, 77, 2237.

Preferably, the preparation of an oxamide by the reaction of carbon monoxide with an amine is carried out in the presence of a catalyst based on a metal from Group VIII of the periodic table, more preferably a platinum group metal, even more preferably palladium.

In a preferred embodiment, said catalyst is ligated with a phosphine-based ligand.

Preferably, the preparation of an oxamide by the reaction of carbon monoxide with an amine is carried out in a solvent selected from the group consisting of acetonitrile, chlorinated solvents such as dichloromethane and chloroform, tetrahydrofuran and hydrocarbyl aromatic solvents such as toluene or benzene. The reaction is also preferably carried out in the presence of a source of iodide ions. Suitably, the source of iodide ions is selected from the group consisting of an alkali metal iodide, or a quaternary ammonium iodide salt. Optionally, a basic compound, such as an alkali metal carbonate or bicarbonate is also added to the reaction.

The preparation of an oxamate in the method of the present invention may be carried out in an analogous method to that described above for the preparation of oxamides. Alternatively, the preparation of an oxamate may be carried out according to the process described in S.-I. Murahashi et al., J. Chem. Soc., Chem. Commun., 1987, 125-127.

Preferably, the preparation of an oxamate is carried out in the presence of a catalyst based on a metal from Group VIII of the periodic table, more preferably a platinum group metal, even more preferably palladium. Optionally, a co-catalyst, such as a metal iodide, preferably copper iodide, is used.

As stated above, the oxamide or oxamate formed in step (i) of the process of the present invention is of general formula (II).

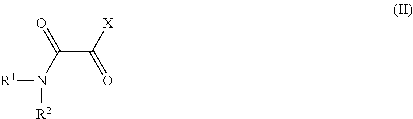

In general formula (II), X is selected from the group consisting of R¹R²N and R³O. R¹ and R² may be the same or different and are functional groups selected from the group consisting of hydrogen and substituted or non-substituted alkyl groups comprising 1 to 8 carbon atoms. Preferably, R¹ and R² are selected from the group consisting of substituted or non-substituted alkyl groups comprising 1 to 8 carbon atoms. The alkyl groups may be linear or branched. Substituted alkyl groups include those substituted with heteroatom containing groups such as hydroxyl groups, ethers and halogens. Alternatively, R¹R²N is a cyclic compound selected from the group of aromatic and non-aromatic cyclic compounds optionally comprising one or more heteroatoms in addition to the nitrogen atom. In this embodiment, aromatic cyclic compounds may contain from 5 to 6 ring atoms and are preferably selected from the group consisting of pyridines, pyrroles, imidazoles, pyrimidines, quinolines, triazoles, oxazoles, thiazoles, pyrazoles, indoles. Non-aromatic cyclic compounds are preferably selected from those containing 5 to 10 ring atoms, more preferably those containing 5 to 8, even more preferably those containing 5 or 6 ring atoms. Optionally, the non-aromatic cyclic compounds may contain one or more heteroatom as well as the nitrogen atom indicated in the formula R¹R²N. Suitably, said heteroatom may be nitrogen, oxygen or sulfur. Preferably, the non-aromatic cyclic compound is selected from the group consisting of piperidines, morpholines and pyrrolidines.

R³ is selected from alkyl groups comprising 1 to 8 carbon atoms. The alkyl groups may be linear or branched and substituted or non-substituted. Substituted alkyl groups include those substituted with heteroatom containing groups such as hydroxyl groups, ethers and halogens. Preferably, R³ is a unsubstituted linear or branched alkyl group comprising 1 to 8 carbon atoms.

In a preferred embodiment of the present invention X is R¹R²N. That is, the compound of general formula (II) is an oxamide. The use of such an intermediate is beneficial as it is lacks the sensitivity to aqueous environments observed during the use of oxalates and, to a lesser extent, oxamates.

In step (ii) the compound of general formula (II) is converted into a compound of general formula (I) by a process that comprises a hydrogenation reaction.

In the compound of general formula (I)

Y—CH₂CH₂—Z (I)

Y and Z are functional groups independently selected from the group consisting of a hydroxyl group and R¹R²N, wherein R¹ and R² and/or R¹R²N are as defined above. Preferably, Y and Z are both hydroxyl groups, i.e. the compound of general formula (I) is monoethylene glycol.

Step (ii) may be carried out by direct hydrogenation of the compound of general formula (II) in order to provide the compound of general formula (I).

Such hydrogenation may be carried out by any suitable hydrogenation method. Preferably, the hydrogenation is catalysed by a catalytic composition based on a metal selected from Group VIII of the periodic table and copper. The metal is preferably platinum, palladium, rhodium, ruthenium, nickel or copper.

Suitably, such hydrogenation is carried out at a temperature in the range of from 100 to 350° C., preferably in the range of from 150 to 300° C. The reaction is typically carried out under a partial pressure of hydrogen in the range of from 100 to 8000 kPa, preferably in the range of from 300 to 7500 kPa.

The conditions of the hydrogenation reaction can be tailored to provide ethylene glycol, ethanolamines and ethyldiamines in the desired ratios.

Alternatively, step (ii) includes the steps of (a) esterifying the compound of general formula (II) to form an oxalate; and (b) reacting said oxalate with hydrogen in the presence of a catalyst.

Step (a) may be carried out under any suitable esterification conditions, including those described in EP 0338386 B1 and T. Itaya et al., Chem. Pharm. Bull, 2002, 346-353. Particularly suitable conditions include reacting the compound of general formula (II) with an alcohol in the presence of a titanium or lead-based catalyst. Preferably, the esterification is carried out at a temperature in the range of from 0 to 300° C., more preferably in the range of from 150 to 250° C. The alcohol may suitably be selected from mono-alcohols containing from 1 to 10, preferably from 1 to 8 carbon atoms.

In step (b) of this embodiment, the oxalate is reacted with hydrogen in the presence of a catalyst. This hydrogenation reaction may be carried out under any suitable hydrogenation conditions, in particular those described in H. T. Teunissen and C. J. Elsevier J. Chem. Soc., Chem. Commun., 1997, 667-668.

In the most preferred embodiment of the present invention, Y and Z are both hydroxyl groups, i.e. the compound of general formula (I) is monoethylene glycol. In this most preferred embodiment, the compound of general formula (II) is an oxamide (i.e. X is R¹R²N). Said oxamide is then hydrogenated directly in order to form the monoethylene glycol. Such a preferred process enables the production of the valuable chemical monoethylene glycol from 1 carbon building blocks (i.e. carbon monoxide) and without using ethylene derivatives in the synthesis. The process also avoids the use of a water-sensitive oxalate intermediate, thus allowing simpler reaction and handling conditions.

The invention will be illustrated by the following non-limiting examples.

### Examples

### General Procedures

Tetramethyloxamide (TMO) was prepared according to the procedure in EP68281B1 using 87.6 g diethyl oxalate (Fluka, 99%) and 192 g 33% dimethylamine/ethanol solution (Fluka).

Bis(morpholino)ethanedione (BMED) was prepared according to the procedure in EP68281B1 using 15.01 g dimethyl oxalate (Sigma-Aldrich, 99%) and 22.11 g morpholine (Merck, 99%).

Oxalic acid diamide (OADA) was purchased from Sigma-Aldrich.

Ethyl-N,N-tetranethyleneoxamate (ETMO) was prepared according to the procedure in EP68281B1 using 14.6 g diethyl oxalate (Fluka, 99%) and 7.1 g pyrrolidine (Fluka, 99%).

The Cu/Al₂O₃/SiO₂ hydrogenation catalyst (‘Cu’) was obtained from KataLuena GmbH Catalysts, while the Pd/Zn/SiO₂ hydrogenation catalyst (‘Pd’) was prepared in an analogous method to the procedure described in U.S. Pat. No. 4,837,368 (example 4) using an impregnation solution of tetraamine palladium(II) nitrate and zinc nitrate.

Titanium(IV) isopropoxide was purchased from Merck and lead(II) oxide from Sigma-Aldrich (99%).

The reaction products were analyzed with NMR and/or GC-MS.

### Esterification

Esterification experiments 1 to 3 were performed by charging substrate, titanium(IV) isopropoxide or lead(II)oxide (see Table 1) and ca. 5 ml 1-octanol into a 25 ml glass flask equipped with a condenser and magnetic stirrer. Then the mixture was stirred and heated to ca. 180° C. Experiment 4 was performed by charging substrate, titanium(IV) isopropoxide and ca. 34 ml ethanol into a 100 ml autoclave equipped with a magnetic stirrer. The autoclave was purged with nitrogen. Then the mixture was stirred and heated to 178° C. After the reaction, the liquid reactor contents were analyzed by GC-MS and/or ¹³C NMR. Table 1 shows the reaction conditions and analytical results from the different experiments.

TABLE 1

| Example | 1 | 2 | 3 | 4 |
|---|---|---|---|---|
| Catalyst | TiO₄C₁₂H₂₈ | PbO | TiO₄C₁₂H₂₈ | TiO₄C₁₂H₂₈ |
| Substrate | TMO | TMO | OADA | TMO |
| Catalyst | ca. 0.15 | n.d. | ca. 0.15 | 0.26 |
| [g] | | | | |
| Substrate | 1.00 | ca. 1 | 0.115 | 0.99 |
| [g] | | | | |
| Alcohol | 1-octanol | 1-octanol | 1-octanol | ethanol |
| T [hr] | 20 | ca. 5 | 23 | ca. 5 |
| Iₙ%¹ | | | | |
| Substrate | 7 | 82 | n.d. | 100 |
| Oxamate | 67 | 10 | n.d. | 0 |
| Oxalate | 26 | 8 | n.d.³ | 0 |
| Aₙ%² | | | | |
| Substrate | 3.6 | | n.d. | 97.6 |
| Oxamate | 66.4 | | n.d. | 2.4 |
| Oxalate | 30.1 | | n.d.³ | 0 |

^{1 13}C NMR carbonyl peak intensity percentage (Iₙ%) = (peak intensity n) × 100/(sum of substrate, intermediate and oxalate peak intensities).
² GC-MS peak area percentage (Aₙ%) = (peak area n) × 100/(sum of substrate, intermediate and oxalate peak areas).
³ This specie was qualitatively observed by NMR analysis and/or GC-MS.
n.d. = not determined

### Hydrogenation

The hydrogenation experiments were performed in a multi-autoclave unit containing four 60 ml batch autoclaves, all equipped with common electrical heating and with individual gas entrainment impellers, manometers and temperature indication. The hydrogenation catalysts were activated in-situ (typical conditions: 230° C., 10-20 bar H₂ for 4 hrs). The substrates, dissolved in ca. 20 ml solvent, were introduced into the autoclaves by injection. Then, the autoclaves were pressurized with H₂, stirred at 800 rpm and heated to ca. 170° C. After the reaction, the liquid reactor contents were analyzed by GC-MS. Table 2 shows the reaction conditions and analytical results from the different experiments.

TABLE 2

| Example | 5 | 6 | 7 | 8 | 9 | 10 | 11 |
|---|---|---|---|---|---|---|---|
| Catalyst | Cu | Pd | Cu | Cu | Cu | Cu | Cu |
| Substrate | TMO | TMO | TMO | TMO | BMED | OADA | ETMO |
| Solvent | methanol | methanol | THF | toluene | methanol | ethanol | ethanol |
| catalyst [g] | 1.22 | 1.13 | 1.16 | 1.21 | 1.24 | 1.20 | 1.21 |
| substrate [g] | 1.50 | 1.41 | 1.44 | 1.45 | 1.94 | 0.308 | ca. 1.67 |
| t [hr] | 17.8 | 18.3 | 18 | 18 | 16.5 | 18 | 18.3 |
| P(H₂) [bar] | 55 | 54 | 55 | 56 | 55 | 49 | 52 |
| Aₙ %⁴ | | | | | | | |
| Substrate | 58.6 | 97.0 | 46.4 | 56.1 | 13.5 | n.d. | 0 |
| HOCH₂(CO)NR₂ | 19.8 | 2.9 | 46.1 | 43.7 | 21.3 | n.d. | 81.5 |
| MEG | 21.6⁵ | 0.2 | 7.5 | 0.3 | 65.2 | n.d.³ | 18.5 |
| HOCH₂CH₂NR₂ | 9.8 | 0.5 | 16.0 | 13.7 | 28.2 | n.d.³ | 22.6 |
| R₂NCH₂CH₂NR₂ | 0.3 | 0.2 | 20.0 | 10.1 | 0 | n.d.³ | 0 |
| ‘polyamines’ | 0 | 0 | 7.5 | 5.4 | 0 | n.d.³ | 0 |

³This specie was qualitatively observed by NMR analysis and/or GC-MS.
⁴GC-MS peak area percentage (Aₙ %) = (peak area n) × 100/(sum of substrate, 2-hydroxyacetamide, MEG, HOCH₂CH₂NR₂, R₂NCH₂CH₂NR₂ and all polyamine peak areas).
⁵MEG peak corrected for overlaying methyl glycolate peak (10% peak area reduction).
n.d. = not determined

The Examples demonstrate a simple process for the production of ethylene glycol, ethanolamines and ethyldiamines from materials obtainable from C-1 building blocks (i.e. carbon monoxide). The process of the present invention is capable of being tailored in order to produce the preferred product(s) and product ratios.

1. A process for the production of compounds of general formula (I),
Y—CH₂CH₂—Z (I)
wherein Y and Z are functional groups independently selected from the group consisting of a hydroxyl group and R¹R²N and wherein R¹ and R² may be the same or different and are functional groups selected from the group consisting of hydrogen and substituted or non-substituited alkyl groups comprising 1 to 8 carbon atoms, or R¹R²N is a cyclic compound selected from the group of aromatic and non-aromatic cyclic compounds optionally comprising one or more heteroatoms in addition to the nitrogen atom, said process comprising the steps of:
(i) reacting carbon monoxide and an amine in the presence of oxygen to provide a compound of general formula II: 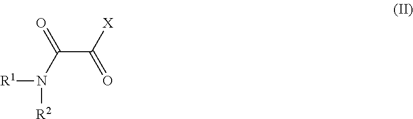
wherein R¹ and R² or R¹R²N are as defined above and X is selected from the group consisting of R¹R²N and R³O, wherein R³ is selected from alkyl groups comprising 1 to 8 carbon atoms; and
(ii) converting the compound of general formula (II) into a compound of general formula (I) by a process that comprises a hydrogenation reaction.

2. A process as claimed in claim 1, wherein X is R¹R²N.

3. A process as claimed in claim 1, wherein both Y and Z are hydroxyl groups or both Y and Z are both R¹R²N.

4. A process as claimed in claim 1, wherein X is R¹R²N and both Y and Z are hydroxyl groups.

5. A process as claimed in claim 4, wherein step (ii) includes the steps of:
(a) esterifying the compound of general formula (II) to form an oxalate; and
(b) reacting said oxalate with hydrogen in the presence of a catalyst.

6. A process as claimed in claim 1, wherein step (ii) is carried out by reacting the compound of general formula (II) with hydrogen in the presence of a catalyst.

7. A process for the preparation of ethylene glycol by the hydrogenation of an oxamide.
